(19) United States
(12) Patent Application Publication
(10) Pub. No.: US 2018/0317825 A1
(43) Pub. Date: Nov. 8, 2018

(54) DEVICE AND METHOD FOR MEASURING THE CONCENTRATION OF A CHEMICAL COMPOUND IN BLOOD

(71) Applicant: BIOSERENITY, Paris (FR)

(72) Inventors: Nadia ARFAOUI, Chatou (FR); Sylvain ZORMAN, Paris (FR); Pierre-Yves FROUIN, Paris (FR)

(21) Appl. No.: 16/065,158

(22) PCT Filed: Dec. 23, 2016
(86) PCT No.: PCT/FR2016/053668

§ 371 Date: Jun. 22, 2018

(30) Foreign Application Priority Data
Dec. 23, 2015 (FR) 1563262

(51) Int. Cl. A61B 5/145; A61B 5/1455

(52) U.S. Cl. CPC A61B 5/14532; A61B 5/14535; A61B 5/14539; A61B 5/14546; A61B 5/14552

(57) ABSTRACT

A device for measuring the concentration of a compound present in the blood includes an adjustable substrate suitable for covering a part of the human body, the adjustable substrate including a light source emitting light beams at at least one wavelength in a range ranging from 700 nm to 3000 nm, the light beams being backscattered by the part of the human body constituting a backscattering source, and at least one photodiode receiver, and a method for measuring the concentration of a chemical compound in blood.

### FIELD OF THE INVENTION

The present invention relates to a device and a method for measuring the concentration of a compound present in the blood by near-infrared spectroscopy.

### STATE OF THE RELATED ART

Within the scope of day-to-day diabetes monitoring in a patient, measuring the blood glucose level is a common procedure and is performed by the patient directly using a medical device. The majority of commercial medical devices intended for diabetes are invasive, i.e. they require piercing of the epidermis to obtain the glucose level measurement. Non-invasive devices have been developed more recently.

As such, the article by Masab Ahmad, Awais Kamboh, Ahmed Khan, Non-invasive blood glucose monitoring using near-infrared spectroscopy, EDN Network, 16 Oct. 2013, discloses a non-invasive device for measuring the glucose level using near-infrared spectroscopy, said device being positioned at the level of the earlobe.

This device comprises five LEDs: two emit light beams at a wavelength of 1550 nm, one emits light beams wherein the wavelength is situated in red, one emits light beams wherein the wavelength is situated in infrared, one light source emits light beams wherein the wavelength is situated qualitatively in green, i.e. it is included in the range of wavelengths ranging from 490 nm to 580 nm and one photodiode having a strong response at 1550 nm.

To determine the blood glucose level, the device uses LEDs emitting light beams wherein the wavelength is situated in red or infrared to measure the glucose and the oxygenation level. Indeed, it is necessary to standardise the glucose level measured with respect to the blood volume at the time of the measurement in order to account for blood volume fluctuations due to cardiac activity. The light source emitting in green makes it possible to measure the thickness of the earlobe, or more generally, the thickness of the zone in question so as to know the distance travelled by the light beam as this determines the absorption of the light beam by the lobe according to a distance-dependent exponential law.

As such, in order to be able to determine the glucose level, it is necessary to consider and determine the blood oxygenation level. The determination of the glucose or oxygenation level is performed in transmittance. This method requires the use of a second light source, in this instance of a diode emitting light beams at a wavelength included in a range ranging from 490 nm to 580 nm.

As such, the measurement of the glucose level cannot be carried out without the use of intermediate measurements. Indeed, it is necessary to measure the oxygenation level and the distance of matter traversed by the light beam. The immediate consequence of these intermediate measurements is a loss in precision due to errors from, on one hand, the thickness measurement and, on the other, errors from the oxygenation determination.

Therefore, there is a need for medical devices exhibiting reliability, reproducibility and precision in the measurements thereof.

Furthermore, this type of device described in the prior art is not suitable for measuring the quantity of other compounds in the blood.

As such, the invention relates to a device and a method enabling the precise determination of the concentration of at least one compound present in the blood without using intermediate measurements such as the determination of a traversed distance and the oxygenation level.

### SUMMARY

The invention relates to a device for measuring the concentration of a compound present in the blood comprising an adjustable substrate suitable for covering a part of the human body, said adjustable substrate comprising:
at least one light source,

said at least one light source emitting light beams at at least one wavelength, said wavelength being included in a range ranging from 700 nm to 3000 nm, said light beams being backscattered by the part of the human body constituting a backscattering source,
at least one photodiode receiver,

said device not comprising diodes emitting light beams at a wavelength included in a range ranging from 490 nm to 580 nm.

According to one embodiment, the at least one light source is an LED or a laser diode. According to one embodiment, the at least one light source is a wavelength-tuneable laser diode.

According to one embodiment, the at least one light source is a white light source.

According to one embodiment, the device comprises means for spectral decomposition of the backscattered light.

According to one embodiment, the measurement device comprises from 2 to 50 light sources.

According to one embodiment, the measurement device comprises from 2 to 2048 photodiode receivers. According to one embodiment, the device comprises a broad-spectrum photodiode receiver.

According to one embodiment, the at least one wavelength of the light beam emitted by the at least one light source is included in at least one of the wavelength ranges centred on wavelengths suitable for measuring the following compounds: total haemoglobin (THb), deoxyhaemoglobin, oxyhaemoglobin, glucose, albumin, lactic acid, triglycerides, and urea.

According to one embodiment, the device is suitable for being arranged around the earlobe, finger, forehead, chin, wrist, foot, hand or neck.

According to one embodiment, the device is suitable for being arranged on an item of clothing in contact with a user's skin.

According to one embodiment, the device may communicate via a wireless system. According to one embodiment, the device comprises wireless communication means.

According to one embodiment, the device further comprises a microcontroller configured to receive information from the at least one photodiode receiver and compute the continuous component and the maximum of the pulsatile component.

According to one embodiment, the device further comprises at least one pair of electrodes suitable for measuring the impedance on the skin surface. According to one embodiment, the electrodes of each pair of electrodes are at a distance of at least 10 cm, 15 cm, 20 cm, 25 cm, 50 cm, 100 cm or 150 cm. According to one embodiment, the device comprises at least two pairs of electrodes. According to one embodiment, the microcontroller is configured to merge the values of the at least one photodiode receiver and the impedance measurement; for example using a Kalman filter.

The invention also relates to a method for measuring the concentration of a compound present in the blood comprising the following steps:
emitting at least one light beam at at least one wavelength included in a range ranging from 700 nm to 3000 nm from at least one light source,
measuring the intensity of the backscattered light as a function of time,
determining the intensity of the backscattered light at the maximum of the pulsatile component and the intensity of the backscattered light at the minimum of the pulsatile component,
computing the concentration of said compound present on the basis of the intensity measured of the at least one backscattered light beam at the minimum and maximum of the pulsatile component.

The invention also relates to the use of the device for simultaneously determining the concentration of various compounds present in the blood, these compounds include but without being limited thereto: total haemoglobin (THb), deoxyhaemoglobin, oxyhaemoglobin, haematocrit, platelets, cholesterol, urea, ammonia, ammonaemia, creatinine, calcium, sodium, potassium, chloride, bicarbonate.

The invention also relates to a system for measuring the concentration of a compound present in the blood comprising an adjustable substrate suitable for covering a part of the human body, said adjustable substrate comprising:
at least one first light source,

said at least one first light source emitting light beams at at least one wavelength, said wavelength being included in a range ranging from 700 nm to 3000 nm, said light beams being backscattered by the part of the human body constituting a backscattering source,
at least one first photodiode receiver suitable for receiving at least a portion of the light beams emitted by the at least one first light source,
at least one second light source,

said at least one second light source emitting light beams at at least one wavelength, said wavelength being included in a range ranging from 700 nm to 3000 nm, said light beams being backscattered by the part of the human body constituting a backscattering source,
at least one second photodiode receiver suitable for receiving at least a portion of the light beams emitted by the at least one second light source,

said adjustable substrate being configured such that, when it covers a part of the human body, the light beams of the at least one first light source are backscattered by a different part of the human body to the part of the human body backscattering the light beams of the at least one second light source.

Definitions
“Pulsatile component”: periodic oscillations over time of light absorption or, conversely, of the intensity of backscattered light of a part of the human body correlated with the variation in arterial blood volume due to cardiac activity. The minimum of the pulsatile component corresponds to the continuous component. The maximum thereof is annotated AC and the minimum thereof is annotated DC.
“Continuous component”: stationary value of light absorption or, conversely, of the intensity of backscattered light of a part of the human body. It is made up of contributions from tissues, bones, venous blood and to the non-pulsatile component of arterial blood. The value thereof is annotated DC.
“LED”: Light-Emitting Diode is an opto-electronic device capable of emitting a non-coherent monochromatic or polychromatic radiations from the conversion of electric energy when a current passes therethrough.
“Laser diode”: opto-electronic device based on semiconductor materials emitting coherent monochromatic light.
“Microcontroller”: integrated circuit including the essential elements of a computer such as the processor, memories, peripheral units and input-output interfaces.

### DETAILED DESCRIPTION

The invention relates to a device for measuring the concentration of a compound present or dissolved in the blood comprising an adjustable substrate suitable for covering a part of the human body, said adjustable substrate comprising:
at least one light source,

said at least one light source emitting light beams at at least one wavelength, said wavelength being included in a range ranging from 700 nm to 3000 nm, said light beams being backscattered by the part of the human body constituting a backscattering source,
at least one photodiode receiver.

According to one embodiment, said device comprises no diodes emitting light beams at a wavelength included in a range ranging from 490 nm to 580 nm.

According to one embodiment, the device comprises at least two light sources, each emitting light beams at a mutually distinct wavelength.

According to one embodiment, the device comprises at least one light source emitting light beams at at least two wavelengths.

According to one embodiment, the at least one light source is an LED or a laser diode.

According to one embodiment, the light source is a white source.

According to one embodiment, the measurement device comprises at least 2 light sources.

According to one embodiment, the measurement device comprises from 2 to 50 light sources.

According to one embodiment, he measurement device comprises from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to 50 light sources.

According to one embodiment, the measurement device comprises a wavelength-tuneable light source. According to one embodiment, the measurement device comprises a single wavelength-tuneable light source such as a wavelength-tuneable laser diode.

According to one embodiment, the at least one light source is a filament lamp. In this embodiment, the wavelength may be varied by varying the intensity of the current passing through the filament lamp. According to one embodiment, the device comprises a single filament lamp.

According to one embodiment, the device may comprise means for spectral decomposition of the backscattered light. According to one embodiment wherein the device comprises means for spectral decomposition of the backscattered light, the at least one light source is a white light source.

According to a further embodiment, the device may further comprise means for spectral decomposition of the backscattered light arranged before the at least one photodiode receiver.

According to one embodiment, the means for spectral decomposition of the backscattered light is a diffraction grating.

According to a further embodiment, the means for spectral decomposition of the backscattered light is a prism or an optical diffraction grating.

According to a further embodiment, the means for spectral decomposition of the backscattered light is made up of at least two filters suitable for the wavelengths emitted.

According to one embodiment, the at least one wavelength of the light beam emitted by the at least one light source is included in at least one of the wavelength ranges centred on wavelengths suitable for measuring the following compounds but without being limited thereto: total haemoglobin (THb), deoxyhaemoglobin, oxyhaemoglobin, haematocrit, platelets, cholesterol, urea, ammonia, ammonaemia, creatinine, calcium, sodium, potassium, chloride or bicarbonate.

According to one embodiment, the at least one wavelength of the light beam emitted by the at least one light source is included in at least one of the wavelength ranges centred on wavelengths suitable for measuring the following compounds but without being limited thereto: total haemoglobin (THb), deoxyhaemoglobin, oxyhaemoglobin, glucose, albumin, lactic acid, triglycerides, water, cholesterol, globulin.

According to one embodiment, the at least one wavelength of the light beam emitted by the at least one light source is included in at least one of the wavelength ranges centred on wavelengths suitable for measuring the following compounds but without being limited thereto: total haemoglobin (THb), deoxyhaemoglobin, oxyhaemoglobin, glucose, albumin, lactic acid, triglycerides, and urea.

For haemoglobin, the suitable wavelength range is from 730 nm to 980 nm.

For deoxyhaemoglobin, the suitable wavelength range is from 730 nm to 805 nm.

For oxyhaemoglobin, the suitable wavelength range is from 805 nm to 980 nm.

For glucose, albumin, lactic acid, triglycerides, cholesterol, globulin or urea, the suitable wavelength range is from 1000 nm to 3000 nm, more specifically, from 2000 nm to 3000 nm and even more specifically, from 2100 nm to 2300 nm.

For water, the suitable wavelength range is from 1 μm to 11 μm; and more specifically from 6 μm to 10 μm and even more specifically from 8 μm to 10 μm.

According to one embodiment, the at least one wavelength of the light beam emitted by the at least one light source is included in at least one of the wavelengths centred on the wavelengths suitable for measuring hormones.

According to one embodiment, the at least one wavelength of the light beam emitted by the at least one light source corresponds to at least one of the wavelengths suitable for measuring the following compounds but without being limited thereto: total haemoglobin (THb), deoxyhaemoglobin, oxyhaemoglobin, glucose, albumin, lactic acid, triglycerides, water, cholesterol, globulin, urea, haematocrit, platelets, cholesterol, ammonia, ammonaemia, creatinine, calcium, sodium, potassium, chloride or bicarbonate.

According to one embodiment, the at least one wavelength of the light beam emitted by the at least one light source corresponds to at least one of the wavelengths suitable for measuring the following compounds but without being limited thereto: total haemoglobin (THb), deoxyhaemoglobin, oxyhaemoglobin, glucose, albumin, lactic acid, triglycerides, water, cholesterol, globulin.

According to one embodiment, the at least one wavelength of the light beam emitted by the at least one light source corresponds to at least one of the wavelengths suitable for measuring the following compounds but without being limited thereto: total haemoglobin (THb), deoxyhaemoglobin, oxyhaemoglobin, glucose, albumin, lactic acid, triglycerides, and urea.

The suitable wavelength for measuring total haemoglobin (THb) is 805 nm.

According to one embodiment, the at least one photodiode receiver is a CMOS or CCD photodiode receiver.

According to one embodiment, the at least one photodiode receiver is a broad-spectrum photodiode receiver. According to one embodiment, the device comprises a single broad-spectrum photodiode receiver. The term broad-spectrum photodiode receiver denotes a photodiode receiver having a sensitive light wavelength response (high signal-to-noise ratio) on the band of light passing through the skin; in particular a photodiode receiver having a sensitive light wavelength response on the band from 700 nm to 10 μm, preferentially from 730 nm to 3000 nm.

According to one embodiment, the measurement device comprises at least 2 photodiode receivers.

According to one embodiment, the measurement device comprises from 2 to 2048 photodiode receivers.

According to one embodiment, the measurement device comprises from 2 to 50 photodiode receivers.

According to one embodiment, the measurement device comprises from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to 50 photodiode receivers.

According to one embodiment wherein the device comprises means for spectral decomposition of the backscattered light, the device comprises a row or an array of photodiodes. According to one embodiment, said row or array of photodiodes comprises from 8 to 2048 photodiodes. According to one embodiment, said row or array of photodiodes comprises from 8, 16, 32, 64, 128, 256, 512, 1024 or 2048 photodiodes.

According to one embodiment wherein the device comprises at least one wavelength-tuneable light source, the device comprises a single broad-spectrum photodiode receiver.

According to one embodiment, the at least one light source is separated from the at least one photodiode receiver by a distance between 0.2 mm and 5 cm, preferentially between 0.5 mm and 3 cm. According to one embodiment, the device comprises at least two light sources and at least two photodiode receivers, the distance separating the first light sources and the first photodiode receiver being different to the distance separating the second light source and the second photodiode receiver. This embodiment makes it possible to obtain information at different depths under the skin. According to one embodiment, the device comprises a plurality of photodiode receivers, the distance between each light source and the corresponding photodiode receiver thereof being progressive so as to analyse the compound over an entire range of subcutaneous depth.

According to one embodiment, the device is characterised by the absence of diodes emitting light beams at a wavelength included in a range ranging from 490 nm to 580 nm. In the prior art, the diode makes it possible to measure the distance travelled by the beam emitted by the light source, this distance being necessary for the application of the Beer-Lambert law in transmittance.

The device according to the invention does away with the need for this distance measurement as it uses a measurement in reflectance based on a ratio between the different intensities measured for at least two compounds such as glucose and Haemoglobin.

According to one embodiment, the adjustable substrate is presented in the form of a coating or an accessory, such as a t-shirt, a headband, a watchstrap.

According to one embodiment, the substrate has elastic or adjustable properties making it possible to apply a mechanical load at the level of the emitters and receivers enhancing the mechanical contact between the emission, reception and skin measurement zone, i.e. the part of the human body in contact with the substrate.

According to one embodiment, the device is suitable for being arranged around the earlobe, finger, forehead, chin, wrist, foot, hand or neck.

According to one embodiment, the device is suitable for being arranged on an item of clothing in contact with a user's skin.

In the following article, Tur et al. Basal Perfusion of the Cutaneous microcirculation: Measurements as a Function of Anatomic Position, The Journal of Investigate Dermatology, Vol. 84, No. 5, pp 442-446, is described the set of anatomic positions whereon spectroscopy measurements may be conducted. In the light of this article, those skilled in the art would know how to apply the device according to the invention to an anatomic position without technical difficulty and without exercising any inventive skill.

According to one embodiment, the device further comprises a system for determining the concentration of said compound present suitable for determining said concentration using backscattered light beams.

This determination system is an electronic device known to those skilled in the art such as a microcontroller.

According to one embodiment, the microcontroller is configured to receive information from the at least one photodiode receiver and extract the continuous component (DC) and the maximum of the pulsatile component (AC) for each of the compounds in question.

This microcontroller enables the use of a specific method for measuring the concentration of a compound present in the blood, this specific method being described hereinafter.

According to one embodiment, the device, in particular the adjustable substrate, also comprises at least one pair of electrodes suitable for measuring the impedance on the skin surface.

According to one embodiment, the impedance measurement makes it possible to measure the concentration of the following compounds but without being limited thereto: total haemoglobin (THb), deoxyhaemoglobin, oxyhaemoglobin, glucose, albumin, lactic acid, triglycerides, water, globulin, urea, haematocrit, platelets, cholesterol, ammonia, ammonaemia, creatinine, calcium, sodium, potassium, chloride or bicarbonate.

According to one embodiment, the impedance measurement makes it possible to measure the concentration of hormones.

According to one embodiment, the electrodes of each pair of electrodes are at a distance of at least 10 cm, 15 cm, 20 cm, 25 cm, 50 cm, 100 cm or 150 cm.

According to one embodiment, the device, in particular the adjustable substrate, comprises at least two pairs of electrodes. In this embodiment, one pair of electrodes is used for injecting a current and one pair of electrodes is used for measurement. This embodiment makes it possible to enhance the precision of the measurement.

According to one embodiment, the microcontroller of the device according to the invention is configured to filter and merge the values of the at least one photodiode receiver and the impedance measurement. According to one embodiment, the filtering and merging are performed by a Kalman filter. This merging and this filtering make it possible to obtain more precise and more robust compound concentration values.

According to one embodiment, the device comprises wireless communication means.

The invention also relates to a method for measuring the concentration of a compound present in the blood comprising the following steps:
emitting at least one light beam at at least one wavelength included in a range ranging from 700 nm to 3000 nm from at least one light source,
measuring the intensity of the backscattered light as a function of time,
determining the intensity of the backscattered light at the maximum of the pulsatile component and the intensity of the backscattered light at the minimum of the pulsatile component,
computing the concentration of said compound present on the basis of the intensity measured of the at least one backscattered light beam at the minimum and maximum of the pulsatile component.

The invention also relates to a method for measuring the concentration of a compound present in the blood comprising the following steps:
emitting at least one light beam at at least one wavelength included in a range ranging from 700 nm to 3000 nm from at least one light source,
measuring the intensity of the backscattered light as a function of time,
determining the intensity of the backscattered light at the minimum of the pulsatile component,
determining the intensity of the backscattered light at the maximum of the pulsatile component,
computing the concentration of said compound present on the basis of the intensity measured of the at least one backscattered light beam at the minimum and maximum of the pulsatile component.

The invention also relates to a method for measuring the concentration of a compound present in the blood comprising the following steps:
emitting at least one light beam at at least one wavelength included in a range ranging from 700 nm to 3000 nm from at least one light source,
measuring the intensity of the backscattered light as a function of time,
determining the intensity of the backscattered light at the maximum of the pulsatile component,
determining the intensity of the backscattered light at the minimum of the pulsatile component,
computing the concentration of said compound present on the basis of the intensity measured of the at least one backscattered light beam at the minimum and maximum of the pulsatile component.

According to one embodiment, the method is implemented with the use of at least two light sources which each emit light beams at at least one mutually distinct wavelength.

According to one embodiment, the method is implemented with the use of at least three light sources which each emit light beams at two wavelengths, said wavelengths being mutually distinct and are included in at least one of the wavelength ranges centred on wavelengths suitable for measuring the following compounds but without being limited thereto, total haemoglobin (THb), deoxyhaemoglobin, oxyhaemoglobin, glucose, albumin, lactic acid, triglycerides, and urea.

According to one embodiment, the method is implemented with the use of at least six light sources which each emit light beams at a wavelength, said wavelengths being mutually distinct and are included in at least one of the wavelength ranges centred on wavelengths suitable for measuring the following compounds but without being limited thereto, total haemoglobin (THb), deoxyhaemoglobin, oxyhaemoglobin, glucose, albumin, lactic acid, triglycerides, and urea.

According to one embodiment, the method is implemented with the use of at least three light sources which each emit light beams at two wavelengths, said wavelengths being mutually distinct and are included in at least one of the wavelength ranges centred on wavelengths suitable for measuring the following compounds but without being limited thereto, total haemoglobin (THb), glucose, albumin, lactic acid, triglycerides, and urea.

According to one embodiment, the method is implemented with the use of at least six light sources which each emit light beams at a wavelength, said wavelengths being mutually distinct and are included in at least one of the wavelength ranges centred on wavelengths suitable for measuring the following compounds but without being limited thereto, total haemoglobin (THb), glucose, albumin, lactic acid, triglycerides, and urea.

According to one embodiment, the method is implemented with the use of at least three light sources which each emit light beams at two wavelengths, said wavelengths being mutually distinct and correspond to the wavelengths of total haemoglobin (THb), deoxyhaemoglobin, oxyhaemoglobin, glucose, albumin, lactic acid, triglycerides, and urea.

According to one embodiment, the method is implemented with the use of at least six light sources which each emit light beams at a wavelength, said wavelengths being mutually distinct and correspond to the wavelengths of total haemoglobin (THb), deoxyhaemoglobin, oxyhaemoglobin, glucose, albumin, lactic acid, triglycerides, and urea.

According to one embodiment, the method is implemented with the use of at least three light sources which each emit light beams at two wavelengths, said wavelengths being mutually distinct and correspond to the wavelengths of total haemoglobin (THb), glucose, albumin, lactic acid, triglycerides, and urea.

According to one embodiment, the method is implemented with the use of at least six light sources which each emit light beams at a wavelength, said wavelengths being mutually distinct and correspond to the wavelengths of total haemoglobin (THb), glucose, albumin, lactic acid, triglycerides, and urea.

In one embodiment, the device and the method described above are suitable for simultaneously determining the concentration of various compounds present in the blood. These compounds include but without being limited thereto: total haemoglobin (THb), deoxyhaemoglobin, oxyhaemoglobin, haematocrit, platelets, cholesterol, urea, ammonia, ammonaemia, creatinine, calcium, sodium, potassium, chloride, bicarbonate.

According to one embodiment, the method comprises a prior calibration step. In this embodiment, the microcontroller is configured to receive an item of information originating for example from a blood test and thereby calibrate the device. In one embodiment, this information is the blood glucose obtained by a blood glucose sensor by pricking a fingertip. In one embodiment, this calibration information is sent wirelessly to the microcontroller via wireless communication means of the device.

The invention also relates to a system for measuring the concentration of a compound present in the blood comprising an adjustable substrate suitable for covering a part of the human body, said adjustable substrate comprising:
at least one first light source,

said at least one first light source emitting light beams at at least one wavelength, said wavelength being included in a range ranging from 700 nm to 3000 nm, said light beams being backscattered by the part of the human body constituting a backscattering source,
at least one first photodiode receiver suitable for receiving at least a portion of the light beams emitted by the at least one first light source,
at least one second light source,

said at least one second light source emitting light beams at at least one wavelength, said wavelength being included in a range ranging from 700 nm to 3000 nm, said light beams being backscattered by the part of the human body constituting a backscattering source,
at least one second photodiode receiver suitable for receiving at least a portion of the light beams emitted by the at least one second light source,

said adjustable substrate being configured such that, when it covers a part of the human body, the light beams of the at least one first light source are backscattered by a different part of the human body to the part of the human body backscattering the light beams of the at least one second light source.

According to one embodiment, the system comprises at least one third light source and at least one third photodiode receiver suitable for receiving at least a portion of the light beams emitted by the at least one third light source; and said adjustable substrate being configured such that, when it covers a part of the human body, the light beams of the at least one first light source, of the at least one second light source and of the at least one third light source are backscattered by a different part of the human body.

According to one embodiment, said system comprises no diodes emitting light beams at a wavelength included in a range ranging from 490 nm to 580 nm.

### BRIEF DESCRIPTION OF THE FIGURES

FIG. 1 represents schematically the progression over time of the intensity received.

### EXAMPLES

The present invention will be understood more clearly on reading the following examples illustrating the invention in a non-limiting manner.

### Example 1

### Determination of Blood Glucose Level

For a given subject and under everyday life circumstances, the blood volume is proportional to the total haemoglobin (THb) concentration. The total haemoglobin (THb) is made up of deoxyhaemoglobin and oxyhaemoglobin. The variation of the blood glucose concentration exhibits a pulsatile component.

In this example, the device comprises at least six light sources each emitting beams at a mutually distinct wavelength. These six (6) wavelengths enable the detection of molecules in the blood which are as follows:
1) λ_{THb} for the detection of total haemoglobin (THb),
2) λ_{Glucose} for the detection of glucose,
3) λ_{Albumin} for the detection of albumin,
4) λ_{LA} for the detection of lactic acid (LA),
5) λ_{Triglycende} for the detection of triglycerides,
6) λ_{Urea} for the detection of urea.

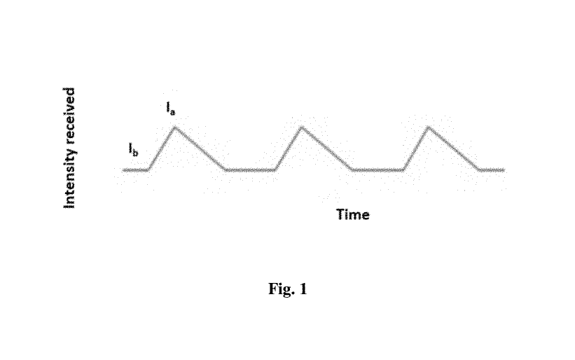

Due to the fact that the variation of the blood glucose concentration exhibits a pulsatile component, the curves obtained have the following format shown in FIG. 1.

As such, according to the modified Beer-Lambert law, it is necessary to solve the following system with six (6) equations and six (6) unknowns:

$$log ( \frac{{I}_{a} ( {λ}_{THb} )}{{I}_{b} ( {λ}_{THb} )} ) = L ( {λ}_{THb} ) * ( {β}_{THb} ( {λ}_{THb} ) * Δ {C}_{THb} + {β}_{Glucose} ( {λ}_{HbT} ) * Δ {C}_{Glucose} + {β}_{Albumin} ( {λ}_{THb} ) * Δ {C}_{Albumin} + {β}_{LA} ( {λ}_{THb} ) * Δ {C}_{LA} + {β}_{Triglyceride} ( {λ}_{HbT} ) * Δ {C}_{Triacetin} + {β}_{Urea} ( {λ}_{THb} ) * Δ {C}_{Urea} )$$

$$log ( \frac{{I}_{a} ( {λ}_{Glucose} )}{{I}_{b} ( {λ}_{Glucose} )} ) = L ( {λ}_{Glucose} ) * ( {β}_{THb} ( {λ}_{Glucose} ) * Δ {C}_{THb} + {β}_{Glucose} ( {λ}_{Glucose} ) * Δ {C}_{Glucose} + {β}_{Albumin} ( {λ}_{Glucose} ) * Δ {C}_{Albumin} + {β}_{LA} ( {λ}_{Glucose} ) * Δ {C}_{LA} + {β}_{Triglyceride} ( {λ}_{Glucose} ) * Δ {C}_{Triglyceride} + {β}_{Urea} ( {λ}_{Glucose} ) * Δ {C}_{Urea} )$$

$$log ( \frac{{I}_{a} ( {λ}_{Albumin} )}{{I}_{b} ( {λ}_{Albumin} )} ) = L ( {λ}_{Albumin} ) * ( {β}_{THb} ( {λ}_{Albumin} ) * Δ {C}_{THb} + {β}_{Glucose} ( {λ}_{Albumin} ) * Δ {C}_{Glucose} + {β}_{Albumin} ( {λ}_{Albumin} ) * Δ {C}_{Albumin} + {β}_{LA} ( {λ}_{Albumin} ) * Δ {C}_{LA} + {β}_{Triglyceride} ( {λ}_{Albumin} ) * Δ {C}_{Triglyceride} + {β}_{Urea} ( {λ}_{Albumin} ) * Δ {C}_{Urea} )$$

$$log ( \frac{{I}_{a} ( {λ}_{LA} )}{{I}_{b} ( {λ}_{LA} )} ) = L ( {λ}_{LA} ) * ( {β}_{THb} ( {λ}_{LA} ) * Δ {C}_{THb} + {β}_{Glucose} ( {λ}_{LA} ) * Δ {C}_{Glucose} + {β}_{Albumin} ( {λ}_{LA} ) * Δ {C}_{Albumin} + {β}_{LA} ( {λ}_{LA} ) * Δ {C}_{LA} + {β}_{Triglyceride} ( {λ}_{LA} ) * Δ {C}_{Triacetin} + {β}_{Urea} ( {λ}_{LA} ) * Δ {C}_{Urea} )$$

$$log ( \frac{{I}_{a} ( {λ}_{Triglyceride} )}{{I}_{b} ( {λ}_{Triglyceride} )} ) = L ( {λ}_{Triglyceride} ) * ( {β}_{THb} ( {λ}_{Triglyceride} ) * Δ {C}_{THb} + {β}_{Glucose} ( {λ}_{Triglyceride} ) * Δ {C}_{Glucose} + {β}_{Albumin} ( {λ}_{Triglyceride} ) * Δ {C}_{Albumin} + {β}_{LA} ( {λ}_{Triglyceride} ) * Δ {C}_{LA} + {β}_{Triacetin} ( {λ}_{Triglyceride} ) * Δ {C}_{Triglyceride} + {β}_{Triacetin} ( {λ}_{Triglyceride} ) * Δ {C}_{Triglyceride} )$$

$$log ( \frac{{I}_{a} ( {λ}_{Urea} )}{{I}_{b} ( {λ}_{Urea} )} ) = L ( {λ}_{Urea} ) * ( {β}_{THb} ( {λ}_{Urea} ) * Δ {C}_{THb} + {β}_{Glucose} ( {λ}_{Urea} ) * Δ {C}_{Glucose} + {β}_{Albumin} ( {λ}_{Urea} ) * Δ {C}_{Albumin} + {β}_{LA} ( {λ}_{Urea} ) * Δ {C}_{LA} + {β}_{Triacetin} ( {λ}_{Urea} ) * Δ {C}_{Triglyceride} + {β}_{Urea} ( {λ}_{Urea} ) * Δ {C}_{Urea} )$$

Where:
Iₐ(λᵢ): intensity measured at the maximum of the pulsatile component at the wavelength i (AC),
I_{b}(λᵢ): intensity measured at the minimum of the pulsatile component at the wavelength i (DC),
L(λᵢ): length of the light path of from the light source to the detector,
Bᵢ: molar absorption coefficient of the compound i,
Cᵢ: blood concentration of the compound i.

Solving the system of equations above makes it possible to find the concentration variations, i.e. the maximum of the pulsatile component, annotated AC, of all the chemical substances in question.

The device described above makes it possible to compute the ratio

$$R = \frac{Δ {C}_{Glucose}}{Δ {C}_{THb}}$$

which makes it possible to determine the blood glucose concentration.

1-20. (canceled)

21. A device for measuring the concentration of a compound present in the blood comprising:
an adjustable substrate suitable for covering a part of the human body,
said adjustable substrate comprising
at least one light source, said at least one light source emitting light beams at at least one wavelength, said wavelength being included in a range ranging from 700 nm to 3000 nm, said light beams being backscattered by the part of the human body constituting a backscattering source, and
at least one photodiode receiver.

22. The device according to claim 21, wherein the at least one light source is an LED or a laser diode.

23. The device according to claim 21, comprising from 2 to 50 light sources.

24. The device according to claim 22, wherein the at least one light source is a wavelength-tuneable laser diode.

25. The device according to claim 21, wherein the at least one light source is a white light source.

26. The device according to claim 25, further comprising means for spectral decomposition of the backscattered light.

27. The device vice according to claim 21, comprising from 2 to 2048 photodiode receivers.

28. The device according to claim 21, comprising a broad-spectrum photodiode receiver.

29. The device according to claim 21, wherein the at least one wavelength of the light beam emitted by the at least one light source is included in at least one of the wavelength ranges centred on wavelengths suitable for measuring the following compounds: total haemoglobin (THb), deoxyhaemoglobin, oxyhaemoglobin, glucose, albumin, lactic acid, triglycerides, and urea.

30. The device according to claim 21, wherein the adjustable substrate is suitable for being arranged around the earlobe, finger, forehead, chin, wrist, foot, hand or neck.

31. The device according to claim 21, wherein the adjustable substrate is suitable for being arranged on an item of clothing in contact with a user's skin.

32. The device according to claim 21, further comprising wireless communication means.

33. The device according to claim 21, further comprising a microcontroller configured to receive information from the at least one photodiode receiver and compute the continuous component and the maximum of the pulsatile component.

34. The device according to claim 21, further comprising at least one pair of electrodes suitable for measuring the impedance on the skin surface.

35. The device according to claim 34, wherein the electrodes of each pair of electrodes are at a distance of at least 10 cm, 15 cm, 20 cm, 25 cm, 50 cm, 100 cm or 150 cm.

36. The device according to claim 34, comprising at least two pairs of electrodes.

37. The device according to claim 33,
further comprising at least one pair of electrodes suitable for measuring the impedance on the skin surface, and
wherein the microcontroller is configured to merge the values of the at least one photodiode receiver and the impedance measurement.

38. A method for measuring the concentration of a compound present in the blood comprising the following steps:
emitting at least one light beam at at least one wavelength included in a range ranging from 700 nm to 3000 nm from at least one light source,
measuring the intensity of the backscattered light as a function of time,
determining the intensity of the backscattered light at the maximum of the pulsatile component and the intensity of the backscattered light at the minimum of the pulsatile component,
computing the concentration of said compound present on the basis of the intensity measured of the at least one backscattered light beam at the minimum and maximum of the pulsatile component.

39. A method for simultaneously determining the concentration of various compounds present in the blood, these compounds include but without being limited thereto: total haemoglobin (THb), deoxyhaemoglobin, oxyhaemoglobin, haematocrit, platelets, cholesterol, urea, ammonia, ammonaemia, creatinine, calcium, sodium, potassium, chloride, bicarbonate, using the device according to claim 21.

40. A system for measuring the concentration of a compound present in the blood comprising an adjustable substrate suitable for covering a part of the human body, said adjustable substrate comprising:
at least one first light source,
said at least one first light source emitting light beams at at least one wavelength, said wavelength being included in a range ranging from 700 nm to 3000 nm, said light beams being backscattered by the part of the human body constituting a backscattering source,
at least one first photodiode receiver suitable for receiving at least a portion of the light beams emitted by the at least one first light source,
at least one second light source,
said at least one second light source emitting light beams at at least one wavelength, said wavelength being included in a range ranging from 700 nm to 3000 nm, said light beams being backscattered by the part of the human body constituting a backscattering source,
at least one second photodiode receiver suitable for receiving at least a portion of the light beams emitted by the at least one second light source,
said adjustable substrate being configured such that, when it covers a part of the human body, the light beams of the at least one first light source are backscattered by a different part of the human body to the part of the human body backscattering the light beams of the at least one second light source.
